(19) United States
(12) Patent Application Publication
(10) Pub. No.: US 2005/0214394 A1
(43) Pub. Date: Sep. 29, 2005

(54) Hippophae rhamnoides compositions for cancer therapy

(76) Inventors: James Dao, Henderson, NV (US); Tom C.S. Dao, Bellevue, WA (US); David D. Tong, Northridge, CA (US)

Correspondence Address: MORRISON & FOERSTER LLP, 755 PAGE MILL RD, PALO ALTO, CA, 94304-1018, US

(21) Appl. No.: 10/948,580

(22) Filed: Sep. 22, 2004

Related U.S. Application Data
(60) Provisional application No. 60/505,053, filed on Sep. 22, 2003.

(51) Int. Cl. A61K 35/78

(52) U.S. Cl. 424/769

(57) ABSTRACT

Methods and compositions for prevention and therapy of cancer using a therapeutically effective amount of an extract of Hippophae rhamnoides (sea buckthorn) leaves, berries, and seeds are provided. Novel uses of these compositions in different stages of cancer therapy are disclosed. Novel compositions comprising Hippophae rhamnoides extracts that preferentially inhibit COX-2 over COX-1 are provided. Compositions comprising therapeutically effective amounts of at least one chemotherapeutic agent in addition to Hippophae rhamnoides are provided.

### CROSS-REFERENCE TO RELATED APPLICATIONS

This application claims the benefit of priority to U.S. Provisional Application No. 60/505,053, filed Sep. 22, 2003 the contents of which is incorporated by reference herein in its entirety. This application is also related to U.S. application Ser. No. ______ (Attorney Docket No. 544302000100), filed Sep. 8, 2004, which is expressly incorporated herein in its entirety.

### TECHNICAL FIELD OF THE INVENTION

This invention relates generally to the field of using extracts of sea buckthorn during the treatment of disease states. More specifically, the invention provides methods and compositions of extracts of sea buckthorn berries and leaves for prevention and therapy of disease states including cancer.

### BACKGROUND OF THE INVENTION

Cancer cells develop because of damage to DNA. Most of the time when DNA becomes damaged, either the cell dies or is able to repair the DNA. In cancer cells, the damaged DNA is not repaired. People can inherit damaged DNA, which accounts for some inherited cancers. Often a person's DNA becomes damaged by exposure to something in the environment, like smoking or exposure to biohazards such as radiation.

Cancer usually forms as a tumor. Some cancers, like leukemia, do not form tumors. Instead, these cancer cells involve the blood and blood-forming organs, and circulate through other tissues where they grow. Cancer cells often travel to other parts of the body where they begin to grow and replace normal tissue. This process, called metastasis, occurs as the cancer cells get into the bloodstream or lymph vessels of our body. When cells from a cancer like breast cancer spread to another organ like the liver, the cancer is still called breast cancer, not liver cancer. Not all tumors are cancerous. Benign (noncancerous) tumors do not spread to other parts of the body (metastasize) and, with very rare exceptions, are not life-threatening.

Any one individual is at risk of developing cancer. The occurrence of cancer increases with aging over a life time (“lifetime risk”). For example, in the U.S., men have a 1 in 2 lifetime risk of developing cancer, and women have a 1 in 3 risk. Other risk factors are believed to include genetics, diet, and environmental exposure (e.g., to mutagenic chemicals, radiation, transforming viruses, etc.). It is estimated by the World Health Organization that about 10 million new cancer cases are occurring now annually around the world. That number is expected to reach 15 million by the year 2015, with two thirds of these new cases occurring in developing countries (World Health 48:22, 1995). For example, it is estimated that there is about 600,000 new cases of lung cancer per year worldwide; approaching 1 million new cases of breast cancer per year; and for head and neck cancer (the sixth most frequently occurring cancer worldwide) an incidence of 500,000 new cases annually. The National Cancer Institute estimates the overall annual costs for cancer at $107 billion. Treatment costs account for approximately $40 billion.

While new therapeutics are being developed and tested for efficacy against tumors, many of the currently available cancer treatments are relatively ineffective. It has been reported that chemotherapy results in a durable response in only 4% of treated patients, and substantially prolongs the life of only an additional 3% of patients with advanced cancer (Smith et al., 1993, J. Natl. Cancer Inst. 85:1460-1474). Many of the current anticancer drugs are both cost-prohibitive, and present with major toxicity. Regarding the latter and depending on the drug or drug combination used, systemic chemotherapy may result in one or more toxicities including hematologic, vascular, neural, gastrointestinal, renal, pulmonary, otologic, and lethal. For example, tamoxifen has been used in women for 25 years to limit breast cancer recurrence. A trial launched in 1992 has shown that tamoxifen is not only effective as a therapeutic agent, but also has a very substantial benefit in cancer prevention (a breast cancer preventative agent). However, in that study, tamoxifen use was shown to have adverse effects in healthy women; i.e., an increased risk of developing uterine cancer or pulmonary blood clots (Science News, 1998, 153:228).

Plants are a valuable resource for the discovery and development of novel, naturally derived agents to treat cancer. Drugs that are currently used in cancer therapy were designed to perturb microtubule shortening (depolymerization) or lengthening (polymerization) (Compton, D. A., et al., (1999) Science 286:913-914). The centrosome, the major microtubule organizing center (MTOC) of the cell, is composed of two centrioles surrounded by the so-called pericentriolar material (PCM), which consists of a complex thin filament network and two sets of appendages (Paintrand, M. (1992) J Struct Biol 108:107-128). The main function of the centrosome is the nucleation of microtubules and the formation of bipolar spindles (Tanaka, T., et al., (1999) Cancer Res 58(17): 3974-85). Centrosomes and their associated microtubules direct events during mitosis and control the organization of animal cell structures and movement during interphase. Malignant tumors generally display abnormal centrosome profiles, characterized by an increase in size and number of centrosomes, by their irregular distribution, abnormal structure, aberrant protein phosphorylation, and by increased microtubule nucleating capacity in comparison to centrosomes of normal tissues (Lingle, W. L. et al., (1998) Proc Natl Acad Sci USA 95(6): 2950-5; Sato. N., et al., (1999) Clin Cancer Res 5(5):963-70; Pihan, G. A. et al., (1998) Cancer Res 58(17):3974-85; Carroll, P. E., et al., (1999) Oncogene 18(11): 1935-44; Xu, X., et al., (1999) Mol Cell 3(3):389-95; Brinkley, B. R., et al., (1998) Cell Motil Cytoskeleton 41(4):281-8; Doxsey, S. (1998) Nat Genet 20(2):104-6; Kuo, K. K., et al., (2000) Hepatology 31(1):59-64). Among the abnormalities, centrosome hyperamplification is found to be more frequent in a variety of tumor types (Carroll, P. E., et al., (1999) Oncogene 18; 18(11):1935-44; Hinchcliffe, E. H., et al., (1999) Science 283(5403):851-4; Xu, X., et al., (1999) Mol Cell 3(3):389-95; Weber, R. G., et al., (1998) Cytogenet Cell Genet 83:266-269).

A variety of drugs currently used in cancer therapy were designed to perturb microtubule polymerization (such as paclitaxel, docetaxel, etoposide, vincristine, vinblastine, and vinorelbine). They share a common mechanism of action of binding to tubulin, the molecule of which microtubules are composed. (Compton, D. A., et al., (1999) Science 286:913-914). At least six plant-derived anticancer agents have received FDA approval (e.g., taxol, vinblastine, vincristine, topotecan, etoposide, teniposide). Other agents are being evaluated in clinical trials (e.g., camptothecin, 9AC, and irinotecan).

For example, taxol, a diterpenoid originally isolated from the bark of the Pacific yew, Taxus brevifolia, is a powerful antimitotic agent that acts by promoting tubulin assembly into stable aggregated structures. (see review Kingston, D. G. I. Trends Biotechnol. 1994, 12, 222; Schiff, P. B.; Fant, J.; Horwitz, S. B. Nature, 1979, 277, 665). Taxol has shown tremendous potential as an anticancer compound. Indeed, it is now used for the treatment of refractory ovarian cancer, and clinical trials are encouraging for the treatment of breast, lung, head, and neck cancers. (Rowinsky, E. K.; Cazenave, L. A.; Donehower, R. C. J. Nat. Cancer Inst. 1990, 82, 1247; McGuire, W. P.; Rowinsky, E. K.; Rosenshein, N. B.; Grumbine, F. C.; Ettinger, D. S.; Armstrong, D. K.; Donehower, R. C. Ann. Int. Med. 1989, 11, 273; Forastiere, A. A., Semin. Oncol. Suppl. 3. 1993, 20, 56).

Chemopreventive agents being investigated for the ability of reducing the amount of pre-cancerous cells in the lungs of smokers and ex-smokers include ACAPHA, a combination of six botanicals (Sophora tonkinensis, Polygonum bistorta, Prunella vulgaris, Sonchus brachyotus, Dictamnus dasycarpus and Dioscorea bulbifera) which has been used for disease prevention in China for centuries. Under a US National Cancer Institute grant, the British Columbia Cancer Agency (Canada) is leading an international consortium in carrying out the phase II clinical trials of ACAPHA.

There is a need for a relatively cost-effective and efficient method for preventing tumors, which additionally ameliorates the toxicity generally associated with systemic chemotherapy and radiation therapy.

Extracts of sea buckthorn (Hippophae rhamnoides) have been used for a variety of purposes. For example, use of unsaturated fatty acids of sea buckthorn seed oil to regulate blood lipids, resist angiocslerosis and radiation, restrains tumour cell, strengthen immunity, and nourishes skin (CN1207920 Zou (1999)); oil from sea buckthorn fruits was claimed to be useful in cosmetic, pharmaceutical, and food products (DE4431393 Lorber and Heilscher (1996)); oil extract of sea buckthorn for skin care products (RU2106859 Senjavina et al. (1998)); sea buckthorn oil in cosmetic cream (RU2134570 Bencharov (1999)); ointment containing sea buckthorn (0.5-1.5%) for suppressing caragenin-induced edemas and passive cutaneous anaphylaxis in patients with inflammatory and allergic skin damages (RU2132183 Prokofet al. (1999)); ointment containing sea buckthom oil for treatment of burns and infected injuries (RU2129423 Frolov (1999)); cosmetic cream containing sea buckthom oil to protect facial skin in winter (RU2120272 Detsina and Selivanov (1998)); cream containing sea buckthom oil showing anti-allergic, bactericidal, anti-inflammatory, and regenerative activities (RU2123320 Chistjakov (1998)).

### SUMMARY OF THE INVENTION

The present invention provides novel compositions, extracts and compounds comprising extracts of sea buckthom (Hippophae rhamnoides) and their methods for manufacture and preparation. Use of such compounds during the prevention and therapy of disease states (such as cancer) are also provided as are methods for preparation and formulation of the compositions as well as methods for treatment using the compositions of this invention. Some embodiments further comprise sea buckthorn with a therapeutically effective amount of at least one chemotherapeutic agent.

General anti-oxidant and immunomodulatory properties of sea buckthom (Hippophae rhamnoides) has been demonstrated previously. The present invention relates to the use of anti-oxidant, immunoboosting and other properties of sea buckthorn for alleviating the toxic effects of chemotherapy and radiation therapy in cancer treatment. The invention also relates to the identification of sources of sea buckthom that display significantly higher anti-oxidant activity. The invention also identifies differences between forms of sea buckthom, such as leaves and berries. Methods for extraction and drying that yield unexpectedly high quality sea buckthom compositions are also disclosed. These methods are claimed for making extracts and preparations of sea buckthom in general.

Synergistic effects of adding sea buckthorn to other botanical extracts are disclosed in U.S. Provisional Application No. 60/501,456, filed Sep. 8, 2003 and incorporated herein by reference. Use of sea buckthom-containing compositions in cancer therapy are provided in this invention, and are based on the disclosed ability of sea buckthorn in reducing toxicity of chemotherapeutic pharmaceuticals.

In one embodiment, compositions of the present invention comprise effective amounts of extracts of Ganoderma lucidum, Scutellaria barbata, Salvia miltiorrhiza, and Hippophae rhamnoides (sea buckthorn) that exhibit cytostatic effects for use in inhibiting further growth of pre-existing cancer cells by exhibiting one or more properties of (i) boosting the immune system, (ii) reducing oxidative damage to cells and tissues, (iii) reducing inflammation, (iv) arresing proliferation of cells in certain stages of the cell cycle, (v) anti-oxidant activity, and (vi) anti-mutagenic effects against further exposure to carcinogens and mutagens.

Compositions of the present invention comprise an effective amount of extracts of Hippophae rhamnoides (sea buckthom) leaves, berries and/or seeds which, by themselves or in combination, pereferentialy inhibit COX-2 enzyme activity over COX-1 activity. In preferred embodiments, an effective amount of H. rhamnoides extract inhibits COX-2 1.5×, 2×, 3×, 5×, or 10× more effectively than COX-1. In some embodiments, an effective amount of H. rhamnoides extract inhibits COX-2 activity and enhances COX-1 activity.

The present invention and other objects, features, and advantages of the present invention will become further apparent in the following Detailed Description of the Invention and the accompanying Figures and embodiments.

### BRIEF DESCRIPTION OF THE FIGURES

FIG. 1 shows an extraction platform for botanical extracts.

FIG. 2 shows extraction procedure with hot water.

FIG. 3 shows extraction procedure with 80% ethanol.

FIG. 4 shows extraction procedure with chloroform/methanol.

FIG. 5A shows the antioxidant components of sea buckthorn berries. FIG. 5B shows the antioxidant components of sea buckthorn leaves.

FIG. 6 shows dose effect curves for Ganoderma lucidum, Salvia miltiorrhiza and Scutellaria barbata (3H) powder blends.

FIG. 7 dose effect curves for Ganoderma lucidum, Salvia miltiorrhiza and Scutellaria barbata (3H) and 3H plus Hippophae rhamnoides (4H) powder blends.

FIG. 8 shows combination index plots for 3H and 4H powder blends.

FIG. 9 shows shows dose effect curves for 3H and 4H hot water extract blends.

FIG. 10 shows combination index plots for 3H and 4H hot water extract blends.

FIG. 11A shows vitamin C content of sea buckthorn and other berries. FIG. 11B shows vitamin E content of sea buckthorn and other berries.

FIG. 12A shows quercetin content of sea buckthorn and other berries. FIG. 12B shows flavonol content of sea buckthorn and other berries.

FIG. 13 shows content of antioxidants of sea buckthorn berries under different drying conditions.

FIG. 14 shows antioxidant activity of botanical blends.

FIG. 15 shows relative contribution of botanicals to antioxidant activity (GL=Ganoderma lucidum; SB=Scutellaria barbata; SL=Salvia miltiorrhiza; SBTL=sea buckthorn leaves).

FIG. 16 shows synergistic effects of botanical extracts administered with anticancer drugs.

FIG. 17A shows the inhibition of COX-2 enzyme activity by different extracts (lipid extract/solvent fraction (LE/SF); lipid extract/water fraction (LE/WF); 80% ethanol (EtOH); and hot water (HW)) of sea buckthorn leaf and berry. FIG. 17B shows the inhibition of COX-1 enzyme activity by different extracts of sea buckthorn leaf and berry.

### DETAILED DESCRIPTION OF THE INVENTION

The present invention provides novel methods and compositions for use as anticancer agents for preventing and treating cancer in an individual. The present invention relates to a novel discovery that botanical extract-based compositions can effectively inhibit tumor growth and be substantially nontoxic when administered to an individual. The compositions comprise two or more extracts of Ganoderma lucidum, Scutellaria barbata, Salvia miltiorrhiza, and optionally, Hippophae rhamnoides (sea buckthorn).

In one embodiment, this method comprises administering a therapeutically effective amount of the composition to an individual (a mammal; and in a preferred embodiment, a human) bearing a tumor. In another embodiment, the method comprises administering a prophylactically effective amount of the composition to an individual to prevent tumor development (e.g., in an individual who is at high risk for developing tumor; or in an individual who is in remission, but at risk for recurrence).

Thus, a primary object of the present invention is to provide a method for treatment of a tumor bearing individual by administering a therapeutically effective amount of a composition having a property of inhibiting tumor growth when administered to the tumor bearing individual.

Another object of the present invention is to provide a method for prevention of tumor development in an individual at risk for tumor development by administering a prophylactically effective amount of a composition having a property of preventing or inhibiting the incidence of tumor growth when administered to the individual.

Another object of the present invention is to provide a method of treatment of a tumor bearing individual, or an individual at risk for developing tumor, with a therapeutically effective amount of a composition that has both properties of inhibiting tumor growth, and being substantially non-toxic when administered to the individual. “Substantially nontoxic” means that the composition lacks the toxicity generally associated with systemic chemotherapy; i.e., lacks detectable toxicities including hematologic, vascular, neural, gastrointestinal, renal, pulmonary, otologic, and immunosuppression (which may lead to lethal infections).

A further object of the present invention is to provide a method of treatment of an individual who has had a substantial reduction in tumor burden but who still is at risk for recurrence, wherein the method comprises administering to the individual a prophylactically effective amount of a composition that has both properties of inhibiting tumor growth, and being substantially non-toxic when administered to the individual.

### DEFINITIONS

“Tumor” is used herein, for purposes of the specification and claims, to mean solid nonlymphoid primary tumor of ductal epithelial cell origin, including, but not limited to, tumors originating in the breast, prostate, colon, lung, pancreas, liver, stomach, bladder, or reproductive tract (cervix, ovaries, endometrium etc.), brain, and bone marrow; melanoma; or lymphoma.

“Inhibiting tumor growth” is used herein, for purposes of the specification and claims, to mean one or more of slowing the growth of the tumor, halting growth of the tumor, causing reduction or regression of the tumor, inhibiting tumor invasion, causing tumor cell death, and causing reduction or regression of metastases.

“Prevention of tumor development” is used herein, for purposes of the specification and claims, to mean inhibiting growth of the tumor; and more specifically, causing tumor cell death in preventing tumor mass formation.

The term “plant” as used herein refers to seeds, leaves, stems, flowers, roots, berries, bark, or any other plant parts that are useful for the purposes described. For certain uses, it is preferred that the underground portion of the plant, such as the root and rhizoma, be utilized. The leaves, stems, seeds, flowers, berries, bark, or other plant parts, also have medicinal effects and can be used for preparing tea and other beverages, cream, and in food preparation.

“Synergism” may be measured by combination index (CI). The combination index method was described by Chou and Talalay. (Chou, T.-C. The median-effect principle and the combination index for quantitation of synergism and antagonism, p. 61-102. In T.-C. Chou and D. C. Rideout (ed.), Synergism and antagonism in chemotherapy. Academic Press, San Diego, Calif. (1991); Chou, T.-C., and P. Talalay. Quantitative analysis of dose-effect relationships: the combined effects of multiple drugs on enzyme inhibitors. Adv. Enzyme Regul. 22:27-55 (1984)). A CI value of 0.90 or less is considered synergistic, with values of 0.85 being moderately synergistic and values below 0.70 being significantly synergistic. CI values of 0.90 to 1.10 are considered to be nearly additive and higher values are antagonistic.

TABLE 1
Synergism/antagonism as a function of CI values

| CI Value | Interpretation |
|---|---|
| >10 | Very strong antagonism |
| 3.3-10 | Strong antagonism |
| 1.45-3.3 | Antagonism |
| 1.2-1.45 | Moderate antagonism |
| 1.1-1.2 | Slight antagonism |
| 0.9-1.1 | Additive |
| 0.85-0.9 | Slight synergism |
| 0.7-0.85 | Moderate synergism |
| 0.3-0.7 | Synergism |
| 0.1-0.3 | Strong synergism |
| <0.1 | Very strong synergism |

It is noted that determination of synergy may be affected by biological variability, dosage, experimental conditions (temperature, pH, oxygen tension, etc.), treatment schedule and combination ratio.

Hippophae rhamnoides (Sea Buckthorn)

Sea buckthorn oil is widely used as a health oil or as a pharmaceutical in Russian and Chinese medicines (Li, T. S. C.; Schroeder, W. R. Sea buckthorn (Hippophae rhamnoides L.): A multipurpose plant. HorTech. 1996, 6, 370-380.). It is reported to prevent liver damage, acute and chronic hepatitis (Xiaoping, T.; Qiaohong, S.; Xiaolan, C.; Jun, C.; Yulan, L.; Qingning, L. Study of biochemical pharmacology of sea buckthorn fruit oil and its compound health products. Proc. Int. Workshop on Sea buckthorn, 1995, Beijing, China, (published)1996, pp 161-164); have therapeutic effects on chemical burns (Nikulin, A. A.; Iakusheva, E. N.; Zakharova, N. M. A comparative pharmacological evaluation of sea buckthorn, rose and plantain oils in experimental eye burns. Eksp. Klin. Farmakol. 1992, 55, 64-66.); and have anti-mutagenic properties (Nersesian, A. K.; Zil'fian, V. N.; Kumkumadzhian, V. A.; Proshian, N. V. Antimutagenic properties of sea buckthorn oil. Genetika 1990, 26, 378-380).

Sea buckthorn seed oil contains a high content of the two essential fatty acids, linoleic acid and α-linolenic acid, which are precursors of other polyunsaturated fatty acids such as arachidonic and eicosa-pentanoic acids. The oil from the pulp/peel of sea buckthorn berries is rich in palmitoleic acid and oleic acid (Chen et al. “Chemical composition and characteristics of sea buckthorn fruit and its oil.” Chem. Ind. Forest Prod. (Chinese) 10 (3), 163-175). The increase in the level of alpha-linolenic acid in plasma lipids showed a clear improving effect on atopic dermatitis symptoms (Yang, B., et al. “Effect of dietary supplementation with sea buckthorn (Hippophae rhamnoides) seed and pulp oils on the fatty acid composition of skin glycerophospholipids of patients with atopic dermatitis.” J Nutr Biochem. Jun. 1, 2000; 11(6):338-340). These effects of α-linolenic acid may have been due to both changes in the eicosanoid composition and other mechanisms independent of eicosanoid synthesis (Kelley 1992, α-linolenic acid and immune response. Nutrition, 8 (3), 215-2).

Sea buckthorn (Hippophae rhamnoides L.) is a rich source of antioxidants both aqueous and lipophilic, as well as polyunsaturated fatty acids. Effects of an antioxidant-rich juice (sea buckthorn) on risk factors for coronary heart disease in humans has been reported. (Eccleston et al. J Nutr Biochem. June 2002; 13(6):346-354.) The effect of sea buckthorn (Hippophae rhamnoides) on cirrhotic patients was investigated and shortening of the duration for normalization of aminotransferases was reported. (Gao Z L. et al., World J Gastroenterol. July 2003;9(7):1615-1617). RH-3, an alcoholic extract of whole berries of Hippopheae rhamnoides, has been demonstrated to provide radioprotective activity in terms of survival of mice against whole body lethal irradiation. (Goel H C, et al. Phytother Res. March 2003;17(3):222-226).

Anti-oxidant and immunomodulatory properties of using sea buckthorn (Hippophae rhamnoides) extracts from powdered leaves and whole berries has been demonstrated using lymphocytes as a model system. (Geetha et al. J Ethnopharmacol March 2002; 79(3):373-8). Cytoprotective activity of sea buckthorn oil has also been reported (Geetha et al., Biomed. Pharmacother. 2002, 56:463-467.) The antiulcerogenic effect of a hexane extract from Hippophae rhamnoides has been demonstrated. (Suleyman H et al., Phytother Res November 2001; 15(7):625-7). Radioprotection by a herbal preparation (30 mg/kg body wt. of mice) of Hippophae rhamnoides berries against whole body lethal irradiation in mice suggested free radical scavenging, acceleration of stem cell proliferation and immunostimulation properties. (Goel H C et al., Phytomedicine January 2002; 9(1):15-25). Inhibition of platelet aggregation by total flavones from sea buckthorn has been reported. (Cheng et al. Life Sciences 2003, 72:2263-2271). Beneficial effects of organic extracts of Hippophae rhamnoides whole berries on nicotine induced oxidative stress in rat blood were compared with Vitamin E (Suleyman H et al. Biol. Pharm. Bull. 25:1133-1136; 2002).

One study found ascorbic acid to be the major antioxidant (approximately 75%) in sea buckthorn juice. (Rosch D. et al., J Agric Food Chem. Jul. 16, 2003;51(15):4233-4239.) Processing effects on the composition of sea buckthorn juice from Hippophae rhamnoides L. Cv. Indian Summer have been reported. (Beveridge T. et al., J Agric Food Chem. Jan. 2, 2002;50(1):113-116).

The effects of H. rhamnoides extracts on apoptosis and cell proliferation appear to be unclear and possibly dependent on the extracts and amounts used. Treatment of mice with 30 mg/kg body wt. of sea buckthorn berry extract increased proliferation in lymphocytes, polymorphs and monocytes. (Goel H C et al. Phytomedicine 9:15-25; 2002). Administration of extract of H. rhamnoides berries to mice before irradiation reduced cellular loss of crypts and villi in jejunum and decreased the frequency of apoptosis in these cells. (Goel H C et al. Phytotherapy Research 17:222-226; 2003). However, the same researchers also found that H. Rhamnoides extracts could generate reactive oxygen species in simple chemical systems and generate DNA-protein cross-links in treated thymocytes. Their study showed differential effects of H. Rhamnoides: free oxygen radicals were produced by cells treated with low concentrations of extract in the absence of radiation while cells treated with high concentrations of extract were able to scavenge free radicals generated by radiation. (Goel H C et al. Molecular and Cellular Biochemistry 245:57-67; 2003). In a concentration-dependent manner, H. Rhamnoides berry extracts induced apoptosis in thymocytes in ex vivo conditions up to 100 μg/ml. However beyond this dose, induction of apoptosis was inhibited. The radioprotective dose of 30 mg/kg body wt. of sea buckthorn berry extract (see above Goel, Phytomedicine 9:15-25; 2002) also induced significant DNA fragmentation in thymocytes. (Goel H C et al. Journal of Environmental Toxicology and Oncology 23:123-137; 2004).

The present invention relates to the use of Hippophae rhamnoides extracts in the prevention of cancer. The anti-oxidant properties of Hippophae rhamnoides are useful in protecting cells from environmental damages to chromosomes and genes and thus reduce the probability of mutations in cancer-related genes.

The present invention also relates to the use of Hippophae rhamnoides extracts in the therapy of cancer. The antioxidant properties of Hippophae rhamnoides are used by co-administration with chemotherapeutic agents. Hippophae rhamnoides reduces the toxic side effects of such agents allow (i) increasing the dosage of chemotherapeutics and/or (ii) reducing the symptoms of administration of chemotherapeutics.

Sea buckthom can be helpful in the treatment of cancer because of its protective effects against radiation therapy and chemotherapy. On the other hand, the strong anti-oxidant properties of sea buckthorn could counteract the cytotoxic effects of agents that prevent proliferation of cancer cells. The compositions of the present invention are prepared to optimize the beneficial effects by adjusting the concentrations of Hippophae rhamnoides extracts.

Compositions

The compositions of the present invention can be in any form which is effective, including, but not limited to dry powders, grounds, emulsions, extracts, and other conventional compositions. To extract or concentrate the effective ingredients of The compositions, typically the plant part is contacted with a suitable solvent, such as water, alcohol, methanol, or any other solvents, or mixed solvents. The choice of the solvent can be made routinely, e.g., based on the properties of the active ingredient that is to be extracted or concentrated by the solvent. Preferred active ingredients of the compositions include, but are not limited to, vitamins C and E, alpha-linolenic acid, phenolocs, phenolic esters, flavonols, anthocyanins, proteins, quercetins, etc. These ingredients can be extracted in the same step, e.g., using an alcoholic solvent, or they may be extracted individually, each time using a solvent which is especially effective for extracting the particular target ingredient from the plant. In certain embodiments, extraction can be performed by the following process: Milling the selected part, preferably leaves, to powder. The powder can be soaked in a desired solvent for an amount of time effective to extract the active agents from the compositions. The solution can be filtered and concentrated to produce a paste that contains a high concentration of the constituents extracted by the solvent. In some cases, the paste can be dried to produce a powder extract of the compositions. The content of active ingredient in the extract can be measured using HPLC, UV and other spectrometry methods.

The compositions of the present invention can be administered in any form by any effective route, including, e.g., oral, parenteral, enteral, intraperitoneal, topical, transdermal (e.g., using any standard patch), ophthalmic, nasally, local, non-oral, such as aerosal, inhalation, subcutaneous, intramuscular, buccal, sublingual, rectal, vaginal, intra-arterial, and intrathecal, etc. It can be administered alone, or in combination with any ingredient(s), active or inactive, including in a medicinal form, or as a food or beverage additive.

In preferred embodiments of the invention, the compositions are administered orally in any suitable form, including, e.g., whole plant, powdered or pulverized plant material, extract, pill, capsule, granule, tablet or a suspension.

The compositions can be combined with any pharmaceutically acceptable carrier. By the phrase, “pharmaceutically acceptable carriers,” it is meant any pharmaceutical carrier, such as the standard carriers described, e.g., Remington's Pharmaceutical Science, 18th Edition, Mack Publishing company, 1990. Examples of suitable carriers are well known in the art and can include, but are not limited to, any of the standard pharmaceutical carriers such as a phosphate buffered saline solutions, phosphate buffered saline containing Polysorb 80, water, emulsions such as oil/water emulsion and various type of wetting agents. Other carriers may also include sterile solutions, tablets, coated tablets pharmaceutical and capsules. Typically such carriers contain excipients such as starch, milk, sugar, certain types of clay, gelatin, stearic acid or salts thereof, magnesium or calcium stearate, talc, vegetable fats or oils, gums, and glycols. Such carriers can also include flavor and color additives or other ingredients. Compositions comprising such carriers are formulated by well known conventional methods. Generally excipients formulated with the compositions are suitable for oral administration and do not deleteriously react with it, or other active components.

Suitable pharmaceutically acceptable carriers include but are not limited to water, salt solutions, alcohols, gum arabic, vegetable oils, benzyl alcohols, gelatin, carbohydrates such as lactose, amylose or starch, magnesium stearate, talc, silicic acid, viscous paraffin, perfume oil, fatty acid monoglycerides and diglycerides, pentaerythritol fatty acid esters, hydroxy methylcellulose and the like. Other additives include, e.g., antioxidants and preservatives, coloring, flavoring and diluting agents, emulsifying and suspending agents, such as acacia, agar, alginic acid, sodium alginate, bentonite, carbomer, carrageenan, carboxymethylcellulose, cellulose, cholesterol, gelatin, hydroxyethyl cellulose, hydroxppropyl cellulose, hydroxypropyl methylcellulose, methylcellulose, octoxynol 9, oleyl alcohol, povidone, propylene glycol monostearate, sodium lauryl sulfate, sorbitan esters, stearyl alcohol, tragacanth, xanthan gum, and derivatives thereof, solvents, and miscellaneous ingredients such as microcrystalline cellulose, citric acid, dextrin, dextrose, liquid glucose, lactic acid, lactose, magnesium chloride, potassium metaphosphate, starch, and the like.

The compositions can also be formulated with other active ingredients, such as anti-oxidants, vitamins (A, C, ascorbic acid, B's, such as B1, thiamine, B6, pyridoxine, B complex, biotin, choline, nicotinic acid, pantothenic acid, B12, cyanocobalamin, and/or B2, D, D2, D3, calciferol, E, such as tocopherol, riboflavin, K, K1, K2). Preferred compounds, include, e.g creatine monohydrate, pyruvate, L-Carnitine, α-lipoic acid, Phytin or Phytic acid, Co Enzyme Q10, NADH, NAD, D-ribose, amino acids such as L-glutamine, Lysine, chrysin; pre-hormones such as 4-androstenedione, 5-androstenedione, 4(or 5-)-androstenediol, 1 9-nor-4 (or 5-)-androstenedione, 1 9-nor-4 (or 5-)-androstenediol, Beta-ecdysterone, and 5-Methyl-7-Methoxy Isoflavone. Preferred active ingredients include, e.g., pine pollen, fructus lycii, Hippophae rhamnoides, Ligusticum, Acanthopanax, Astragalus, Ephedra, codonopsis, polygola tenuifolia Willd, Lilium, Sparganium, ginseng, panax notogiseng, Garcinia, Guggle, Grape Seed Extract or powder, and/or Ginkgo Biloba.

Other plants and botanicals which can be formulated with the compositions of the present invention includes those mentioned in various text and publications, e.g., ES Ayensu, Medicinal Plants of West Africa, Reference Publications, Algonac, Mich. (1978); L. Boulos, Medicinal Plants of North Africa, Reference Publications Inc., Algonac, Mich. (1983); and N. C. Shah, Botanical Folk Medicines in Northern India, J. Ethnopharm, 6:294-295 (1982).

Other active agents include, e.g., antioxidants, anti-carcinogens, anti-inflammatory agents, hormones and hormone antagonists, antibiotics (e.g., amoxicillin) and other bacterial agents, and other medically useful drugs such as those identified in, e.g., Remington's Pharmaceutical Sciences, 18th Edition, Mack Publishing Company, 1990. A preferred composition of the present invention comprises, about 1%-100%, preferably about 20-70% of the botanical extract; and, optionally, a pharmaceutically-acceptable excipient.

The present invention relates to methods of administering the compositions, e.g., to provide antioxidant effects, to protect against oxidation, to provide anti-cancer effects, to promote DNA repair, to provide anti-radiation effects, to protect against radiation, to reduce inflammation, and other conditions and diseases as mentioned herein.

By the term “administering,” it is meant that the compositions are delivered to the host in such a manner that it can achieve the desired purpose. As mentioned The compositions can be administered by an effective route, such as orally, topically, rectally, etc. The compositions can be administered to any host in need of treatment, e.g., vertebrates, such as mammals, including humans, male humans, female humans, primates, pets, such as cats and dogs, livestock, such as cows, horses, birds, chickens, etc.

An effective amount of the compositions are administered to such a host. Effective amounts are such amounts which are useful to achieve the desired effect, preferably a beneficial or therapeutic effect as described above. Such amount can be determined routinely, e.g., by performing a dose-response experiment in which varying doses are administered to cells, tissues, animal models to determine an amount effective in achieving a desired result. Amounts are selected based on various factors, including the milieu to which the composition is administered (e.g., a patient with cancer, animal model, tissue culture cells, etc.), the site of the cells to be treated, the age, health, gender, and weight of a patient or animal to be treated, etc. Useful amounts include, 1-100, 5-500, 10-1000 μg/mL, 10 milligrams-100 grams, preferably, e.g., 100 milligrams-10 grams, 250 milligrams-2.5 grams, 1 gm, 2 gm, 3 gm, 500 milligrams-1.25 grams or higher, per dosage of different forms of the compositions such as the botanical powder, botanical extract paste or powder, tea and beverages prepared to contain the effective ingredients of the compositions, and injections, depending upon the need of the recipients and the method of preparation.

Therapeutic Hippophae rhamnoides Compositions

The invention relates to compositions comprising Hippophae rhamnoides (sea buckthorn) extracts that are effective in “early stage” cancer and pre-cancerous conditions by exhibiting one or more properties of (i) boosting the immune system, (ii) reducing oxidative damage to cells and tissues and (iii) anti-inflammatory activity such as COX-2 inhibition. Hippophae rhamnoides (sea buckthorn) extracts and other anticancer compounds such as chemotherapeutic agents are included in a typical composition.

Chemotherapeutic agents suitable for use in the compositions and methods of the present invention may be any known pharmaceutically acceptable agent that depends, at least in part, on interfering with cellular structure and/or metabolism for its anticancer activity. Examples of conventional chemotherapeutic agents include, but are not limited to, platinum compounds such as cisplatin, carboplatin and their analogs and derivatives; alkylating agents such as chlorambucil, nitrogen mustards, nitromin, cyclophosphamide, 4-hydroperoxycyclophosphamide; 2-hexenopyranoside of aldophosphamide, melphalan, BCNU, CCNU, methyl-CCNU, uracil mustard, mannomustine, triethylenemelamine, chlorozotocin, ACNU, GANU, MCNU, TA-77, hexamethylmelamine, dibromomannitol, pipobroman, epoxypropidine, epoxypiperazine, ethoglucide, pippsulfan, dimethylmilelane, bubulfan, inprocuon, threnimone, thio-TEPA and Aza-TEPA; antimetabolites such as 5-fluorouracil, folic acid, methotrexate (MTX), 6-mercaptopurine, aminopterin, 8-azaguanine, azathioprine, uracil, cytarabine, azaserine, tegaful, BHAC, SM108, cytosine arabinoside, cispuracham, diazamycine, HCFU, 5′DFUR, TK-177 and cyclotidine; antibiotics such as bleomycin, daunomycin, cyclomycin, actinomycin D, mitomycin C, carzinophylin, macrocinomycin, neothramycin, macromomycin, nogaromycin, cromomycin, 7-o-methylnogallol-4′-epiadriamycin, 4-demethoxydaunorubicin, streptozotocin and mitozanthron; bis-chloroethylating agents, such as mafosfamide, nitrogen mustard, nornitrogen mustard, melphalan, chlorambucil; hormones such as estrogens; bioreductive agents such as mitomycin C and others such as mitoxantrone, procarbazine, adriblastin, epirubicin, prednimustine, ifosfamid, P-glycoprotein inhibitors such as thaliblastine and protein kinase inhibitors such as protein kinase C inhibitor (ilmofosine). Chemotherapeutic agents particularly refer to the antimicrotubule agents or tubulin targeting agents including vinca alkaloids such as etoposide, podophyllotoxin, vincristine and vinblastine; taxanes (paclitaxel, docetaxel and precursor taxane (10-deacetylbaccatin III), arsenic salts, colchicin (e), thio-colchicine, coichiceine, colchisal and other colchium salts; epipodophyllotoxins (etoposide), cytochalasins (such as A-E, H, J), okadaic acid, carbaryl and it's metabolites such as naphthol or naphthyl compounds including 1-naphthol, 2-naphthol, 1-naphthylphosphate, malonate, nocodazole (methyl-(5-[2-thienyl-carbonyl]-1H-benzimidazol-2-yl)carbamate), cryptophycin (CP) and its analogues such as CP-52, wortmannin, 12-0-tetradecanoylphorbol-1 3-acetate (TPA), 14-3-3 sigma and its homologs (such as rad24 and rad25), Ustiloxin F, monocrotalines such as monocrotaline pyrrole (MCTP), estramustine and the inhibiting agents of adenosine. These chemotherapeutic agents may be used either alone or in combination. Preferably, one antimetabolite and one antimicrotubule agent are combined, and more preferably taxol, cisplatin, chlorambucil, cyclophosphamide, bleomycin, or 5-fluorouracil which have different tumor killing mechanisms are combined. The combination containing arsenic compounds, colchicin, colchicine, colchiceine, colchisal, colchium salts, vinblastine, paclitaxel and related compounds that interfere with the cytoskeletons are most preferred. As new chemotherapeutic agents and drugs are identified and become available to the art, they may be directly applied to the practice of the present invention.

In a preferred embodiment, an all natural composition comprises H. rhamnoides extracts in combination with plant components such as cyclophosphamide, 4-hydroperoxycyclophosphamide, thiotepa, taxol and related compounds, doxorubicin, daunorubicin and neocarzinostain in addition to one or more extracts of Ganoderma lucidum, Scutellaria barbata, and Salvia miltiorrhiza.

Drugs that are currently used in cancer therapy and designed to perturb microtubule shortening (depolymerization) or lengthening (polymerization) such as paclitaxel, docetaxel, etoposide, vincristine, vinblastine, and vinorelbine are a component of Hippophae rhamnoides compositions. These drugs bind to tubulin, the molecule of which microtubules are composed, and arrest cells in mitosis by inhibiting spindle assembly (Compton, D. A., et al., (1999) Science 286:313-314).

The methods according to the present invention for anticancer therapy with Hippophae rhamnoides compositions further comprises administering a therapeutically effective amount of one or more standard anticancer treatments (e.g., one or more of radiation therapy, chemotherapy, surgery, immunotherapy, and photodynamic therapy) in addition to administering a therapeutically effective amount of the composition. In a preferred embodiment of this alternative, the method comprises administering a therapeutically effective amount of one or more standard chemotherapeutic drugs in addition to administering a therapeutically effective amount of the composition. A combination of a therapeutically effective amount of one or more standard chemotherapeutic drugs and a therapeutically effective amount of the composition of Hippophae rhamnoides, allows use of larger doses of the chemotherapeutic due to the alleviation of its toxic side effects by Hippophae rhamnoides.

The invention also relates to compositions comprising Hippophae rhamnoides (sea buckthorn) extracts that treat “advanced stage” cancer by exhibiting one or more properties of (i) boosting the immune system, (ii) reducing oxidative damage to cells and tissues, and (iii) increasing tolerance to standard therapies. In a preferred embodiment, hot water extracts of Hippophae rhamnoides (sea buckthorn) are used. Extracts, especially hot water extracts of Hippophae rhamnoides exhibit significant anti-oxidative properties and increased tolerance to standard chemotherapies and radiation therapy.

### EXAMPLES

Without further elaboration, it is believed that one skilled in the art can, using the preceding description, utilize the present invention to its fullest extent. The following examples are illustrative only, and not limiting of the remainder of the disclosure in any way whatsoever.

In addition to Hippophae rhamnoides, the following botanical extracts were used throughout the examples: Panax quinquefolium (Western ginseng), Ganoderma lucidum, Scutellaria barbata, Salvia miltiorrhiza and Camellia sinensis (green tea).

Results obtained with these combinations or the individual extracts were often compared with ACAPHA, a combination of six botanicals (Sophora tonkinensis, Polygonum bistorta, Prunella vulgaris, Sonchus brachyotus, Dictamnus dasycarpus and Dioscorea bulbifera).

### Example 1

### Methods for Preparation of Botanical Extracts

The compositions of the present invention may be administered as dried botanicals. Botanical preparations contain phytochemicals some of which are soluble in aqueous media while others are relatively more soluble in organic (alcohol, lipid) media. Different extraction methods were used and tested for the ability to extract effective ingredients from the botanicals. Extraction methods include: Hot Water-extraction; Organic (lipid or solvent fraction) extraction; Organic (aqueous fraction) extraction; and Ethanol Extraction.

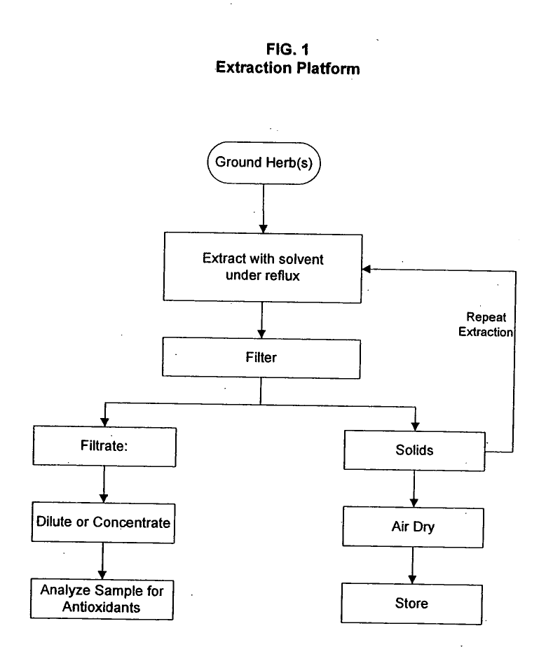

Products are prepared from botanicals using different solvents by the general extraction platform shown in FIG. 1. Botanical or botanical blends were extracted with solvent (hot water, 80% ethanol, or chloroform/methanol) under reflux for 30-60 minutes, separated by filtration to obtain a filtrate, and air dried for further analysis. The filtrates were combined, diluted or concentrated prior to determination of activities. Preferably, the extraction is repeated more than once, however recovery tended to be low in the third extraction.

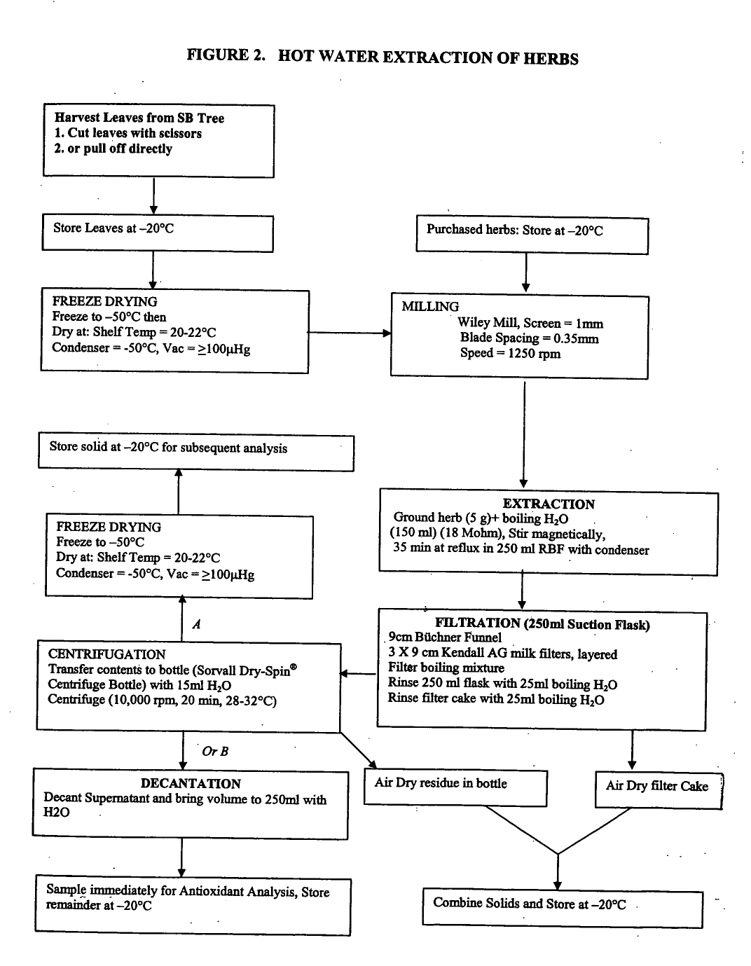

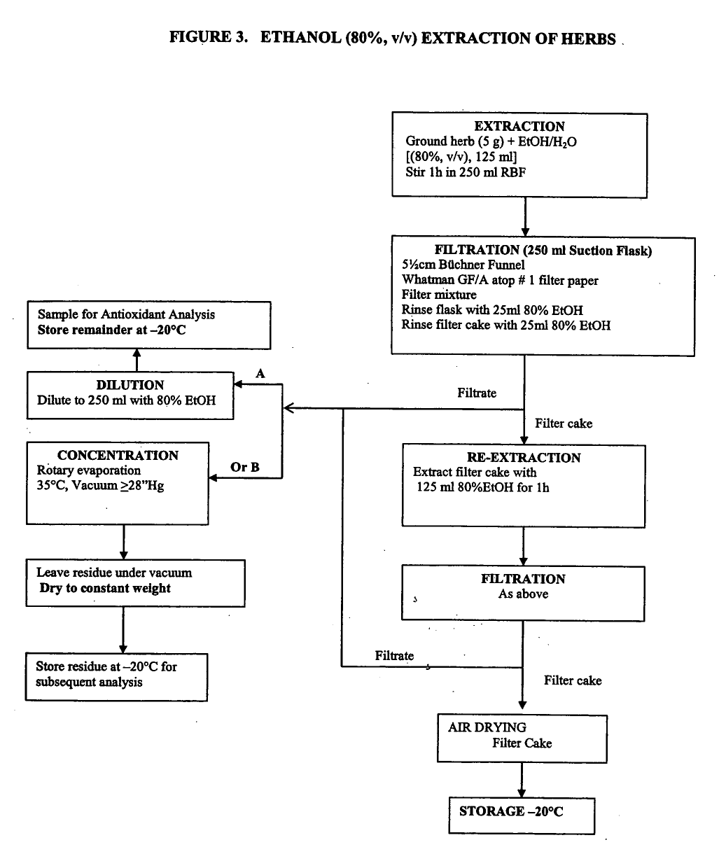

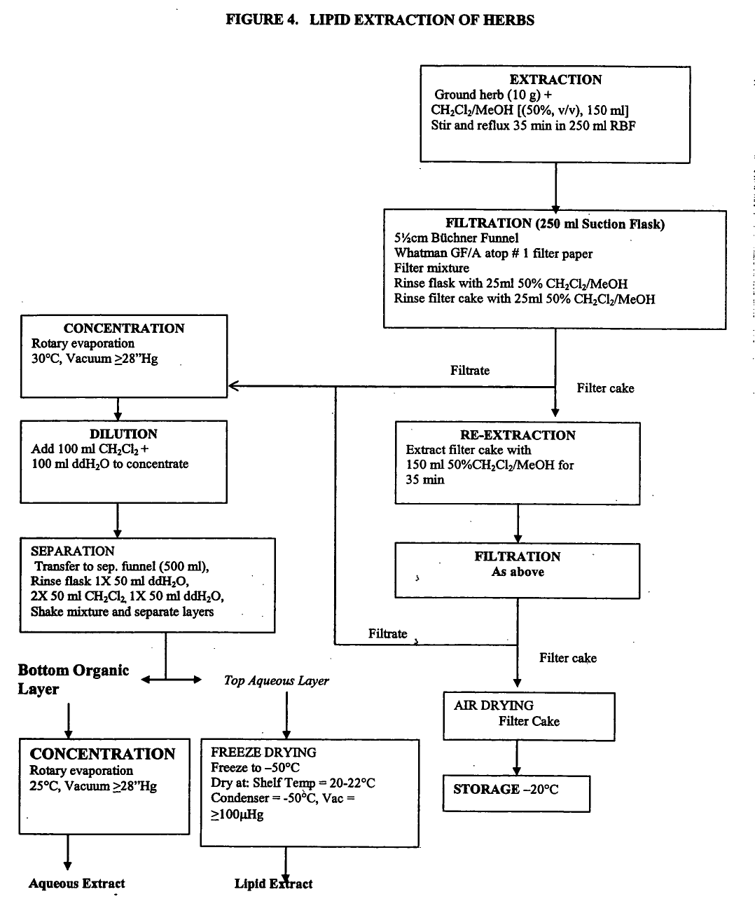

Extraction procedures with hot water, 80% ethanol and chloroform/methanol are shown schematically in FIGS. 2, 3 and 4 respectively. In general, hot water extracts of botanicals have the highest concentrations of phenolics, phenolic esters, flavonols and anthocyanins. Sea buckthorn berry and leaf have high concentrations of these ingredients. In one embodiment sequential hot water (1×) followed by ethanol extraction was most suitable for extraction of flavonoids.

### Example 2

### Properties of Sea Buckthorn Leaf and Berry Extracts

Weight, size and yield of berries, and seeds vary significantly among cultivars of sea buckthorn, such variation also evident seasonally. Physicochemical characteristics of sea buckthorn are cultivar dependant even when grown in one location. Depending on cultivar the juice yield varies from 73% to 91% and soluble solids range from 7.7 to 15.2 °Brix. Similarly ascorbic acid content and the total carotenoid content in juice, also vary from 31 to 754mg/100 g and 7 to 19 mg/100 g of fruit, respectively. Significant differences among cultivars were also observed in antioxidant efficiency (AE) of juice which ranged from 9.5% to 88%.

The seed oil content, extracted with hexane, ranges from 9.1% to 15.5% and that of the fruit pulp oil varies from 29% to 49%, depending on cultivar. Results of tocopherol analysis show that the vitamin E content is also cultivar dependant and vary from 106 to 161 mg/100 g in seed oil and 76 to 227 mg/100 g in fruit pulp oil.

Moisture content of whole berry samples were determined by a single stage air oven method (60° C./24 h). Thoroughly mixed juice samples were centrifuged at 5000 rpm for 15 min and aliquots from the clear juice fraction of each sample were taken to determine the soluble solid content of juice using an Abbe digital refractometer (Mark II type).

Oil from seeds were extracted with hexane (1:5 w/v ground seeds to hexane) for 3h, the hexane evaporated and oil content was measured gravimetrically. Pulp was obtained from juice by centrifugation at 4° C. at 15000 rpm for 15 min, then stored at −25° C. for 2 h. The top layer was used as the pulp; oil was recovered by homogenizing the pulp with hexane (1:1 w/v) and measured gravimetrically.

Tocopherols were determined by HPLC using known methods. (Bourgeois, C. 1992. Determination of Vitamin E: Tocopherols and Tocotrienols. Elsevier Applied Science, London and New York). Antioxidant efficiency (AE), defined as the percent relative activty of a sample compared to that of butylated hydroxytoluene (BHT), was determined by the beta-carotene method. (Velloglu Y S, Mazza G, Gao L and Oomah B D, Antioxidant activity and total phenolics in selected fruits, vegetables and grain products. J Agric Food Chem 46:4113-4117 (1998))

Total carotenoid content of fruit juice, seed oil and pulp oil were determined using a scanning UV-vis spectrophotometer (Beckman DU-600).

Ascorbic acid contents of juice samples from different cultivars were determined by HPLC according to a modified method described by Acar and Gokman (1996). (Gokman, V and Acar, J A Simple HPLC method for determination of total vitamin C in fruit juices and drinks. Fruit Processing 5:198-201).

Determination of antioxidant activity is based on the ABTS radical cation decoloration assay adapted for microplates. Extract sample solutions are prepared in distilled water to a range of concentrations representing 0-100 mg/L. The method is based on the measurement of relative radical-scavenging capacities of extracts containing flavonoids and phenolics through their properties as electron or proton donating agents. (Pellegrini, N.; Re, R.; Yang, M.; Rice-Evans, C. 1999. Meth. Enzym. 299, 379-389.). Upon interaction of antioxidants with ABTS (2,2′-azinobis(3-ethyenebenzothiazoline-6-sulfonic acid)) free radicals, the radical is reduced and its green color suppressed to an extent on a time scale. The reduction rate of free radicals is measured as decrease in absorbance at 734 nm. Relative antioxidant capacity is measured in the presence of Trolox or Quercetin standards and expressed as trolox (or quercetin) equivalent antioxidants present per dry gram of botanical.

In one embodiment, ABTS stock solution was prepared by mixing 5 ml of 7 mM ABTS [2,2′-azinobis(3-ethylbenzothiazoline-6-sulfonic acid)] with 88 μl of 140 mM K₂S₂O₈. The stock solution was diluted with ethanol to give an absorbance at 734 nm of 0.7±0.05 (Pellegrini et al. 1999). The extract of sea buckthorn (100 μl of 20 mg/ml) was mixed with 1 ml ABTS reagent and measured at 734 nm after 30 min in room temperature. The absorbance difference between aqueous and phenolic (ascorbate-free) extracts corresponded to ascorbic acid. Trolox was used as a standard and the capacity of free radical scavenging was expressed as trolox equivalent mg/g of antioxidant capacity.

Sea buckthorn leaf and berry extracts are significant sources of vitamins A, C, E, K and pantothenic acid. Vitamin K influences the synthesis of interleukins 1 and 6; vitamin C lowers the prevalence of Heliobacter pylori infection thereby reducing the risk of peptic ulcers and stomach cancer. Smoking lowres serum levels of vitamin C and somkers are advised to supplement vitamin C intake. Vitamins A, C and E are antioxidants which protect the human body from oxidative damage leading to cancer, heart disease and aging.

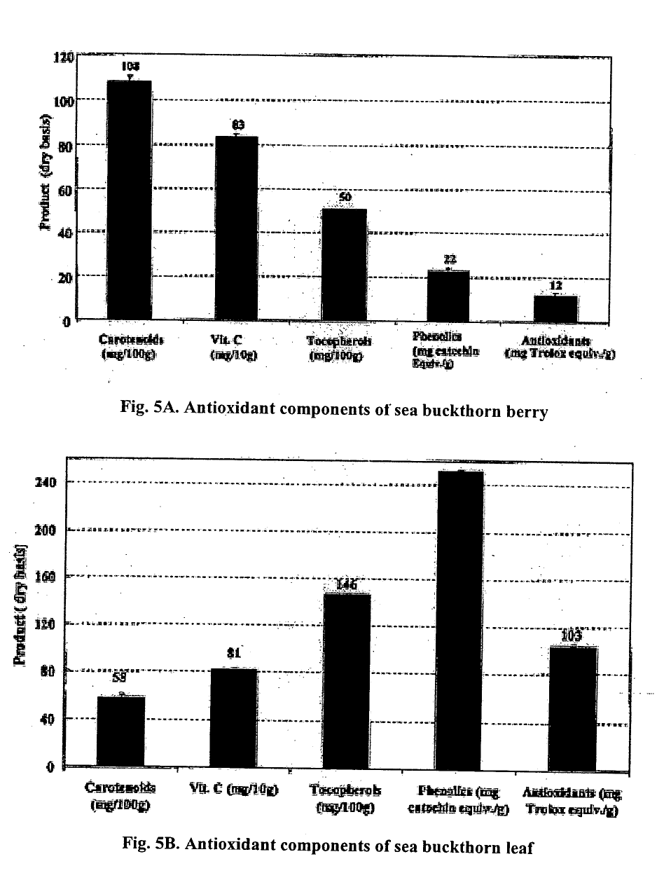

As shown in FIGS. 5A and 5B, the leaf and berry have significantly different profiles of antioxidant activity. Beta-carotene, vitamins C and E (tocopherols) contribute significantly to the antioxidant activity of the berry while phenolics play a minor role. The leaves show significantly (more than 5×) antioxidant activity due to the high content of phenolics and tocopherol and moderate levels of vitamin C with carotenoids having a minor effect.

### Example 3

### Synergism in Activities of Sea Buckthorn and Botanical Extracts

Blends of botanical extracts comprising Ganoderma lucidum, Salvia miltiorrhiza and Scutellaria barbata (3H) and optionally sea buckthorn berry and/or leaf (4H) were tested for anti-oxidant property. Antioxidant activity was measured as described above in trolox and quercetin equivalents. In addition, the phenolic antioxidant index (PAOXI), a combined measure of quality and quantity of antioxidants, was measured by dividing the total phenolic concentration by its ED₅₀ value. (Vinson et al. J. Agric. Food Chem. 46:3630-3634 (1998)). Blending the botanicals before extraction increased the PAOXI values for both 3H and 4H extracts. PAOXI values for hote water (HW) and lipid extract/water fraction (LE/WF) of 4H blends were higher than those of 3H blends.

The trolox equivalent antioxidant capacity (TEAC) assay indicated that Salvia miltiorrhiza was the primary contributor of antioxidant activity for the HW and LE.WF exracts. In lipid extract/solvent fraction (LE/SF) Salvia miltiorrhiza contributed the least and Scutellaria barbata (59%) and Ganoderma lucidum (27%) contributed significantly to the antioxidant potential of the 3H extracts. Sea buckthorn leaf was found to be responsible for nearly 50% on the anti-oxidant activity of the entire blend under both systems of measurement for the 4H extracts.

Antioxidant activity data for the 3H and 4H extracts at different concentrations were statistically analysed using CalcuSyn for Windows software (T-C Chou and P. Talalay (Trends Pharmacol. Sci. 4, 450-454)) to dtermine whether the botanical combinbations were additive, synergistic or antagonistic. Dose-reduction index (DRI) meaures by how much the dose of a botanical in a synergistic combination may be reduced at a given effect level compared to each botanical administered singly. Combination index (CI) is a quantitative measure of the degree of interaction interms of additive effect (CI=1), synergism (CI<1) or antagonism (CI>1) for a given point of effect.

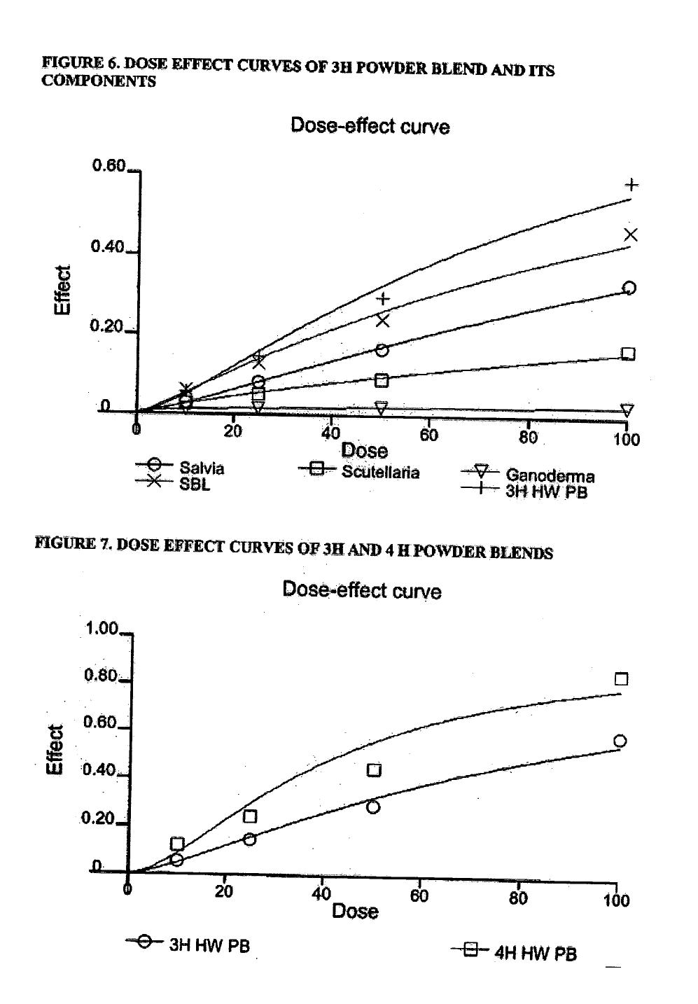

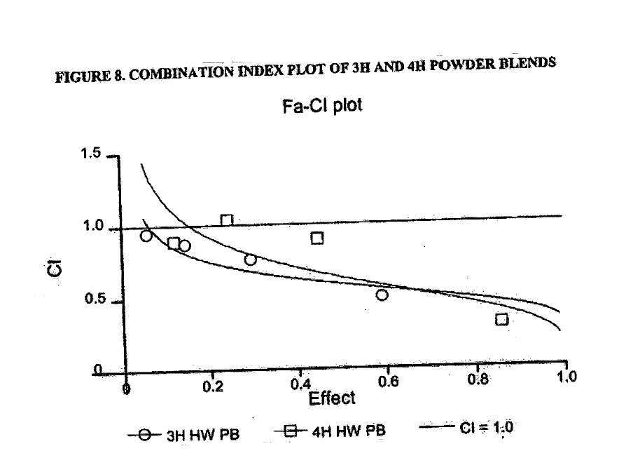

Free radical scavenging activity (% inhibition) of different concentrations of hot water extracts of individual botanicals and 3H and 4H powder blends were measured. The slopes of plots for single botanicals range from 0.855 to 1.584 suggest they have similar mode of action as shown in FIG. 6. The extremely low activity of G. lucidum may reflects low solubitity in hot water. FIG. 7 shows the effect of 4H blend (with sea buckthorn) is significantly higher than the 3H blend at a particular dose.

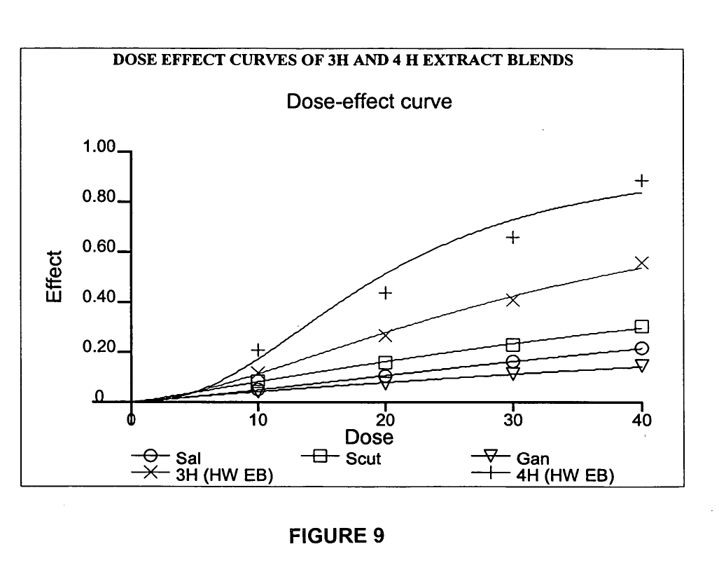

Both 3H and 4H powder blends at ED₅₀ or higher doses showed synergistic effects with CI values between 0.3-0.7 as shown in FIG. 8, i.e. the observed antioxidant activities were higher than expected.

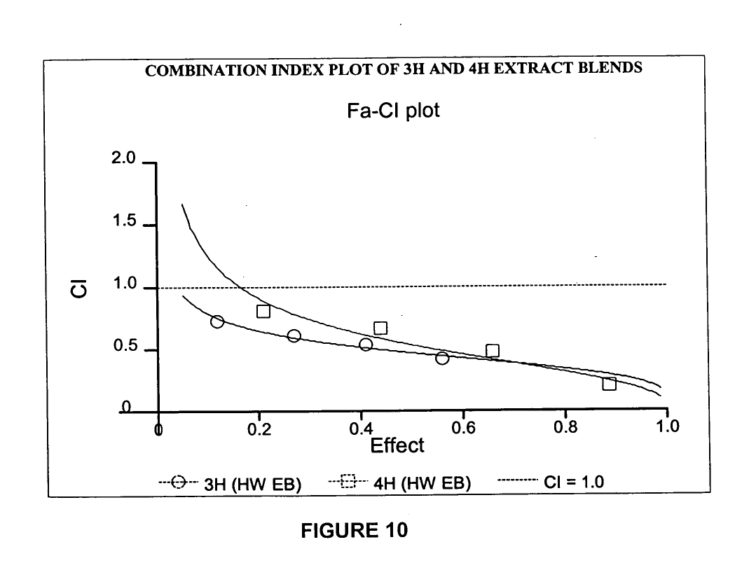

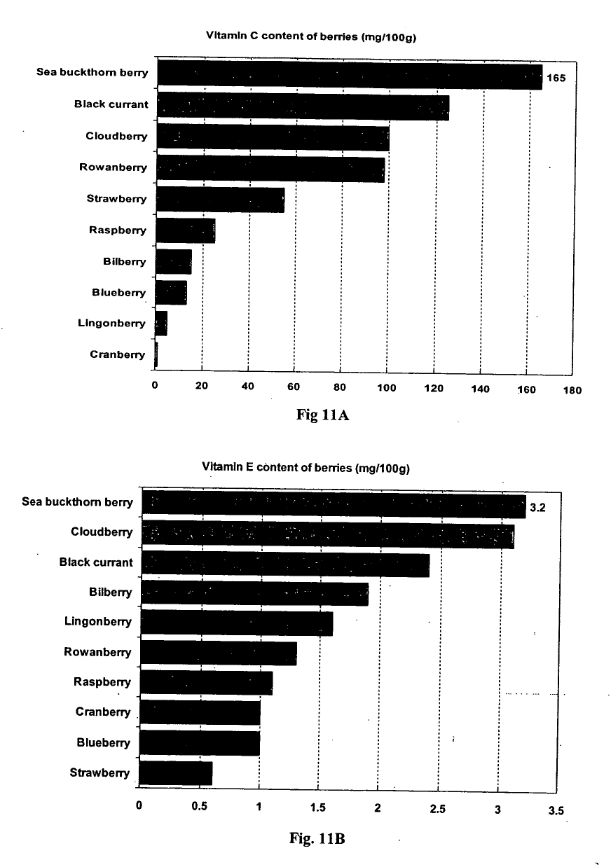

Unlike the powder blends, the dose reduction index of hot water extracts inedicated that G. lucidum was necessary for the combination to be effective. Similar to the powder blends, both 3H and 4H hot water extracts showed synergistic effects with CI values between 0.2 and 0.6 (FIGS. 9 and 10). The 4H blend (with sea buckthorn) has higher synergistic effect than the 3H blend as shown in FIG. 10.

### Example 4

### Comparison of Sea Buckthorn with Other Berries

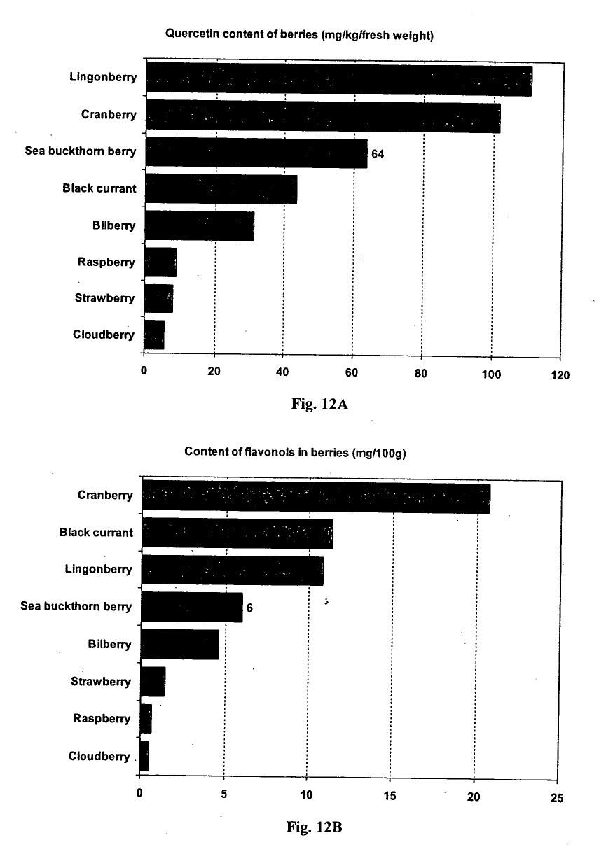

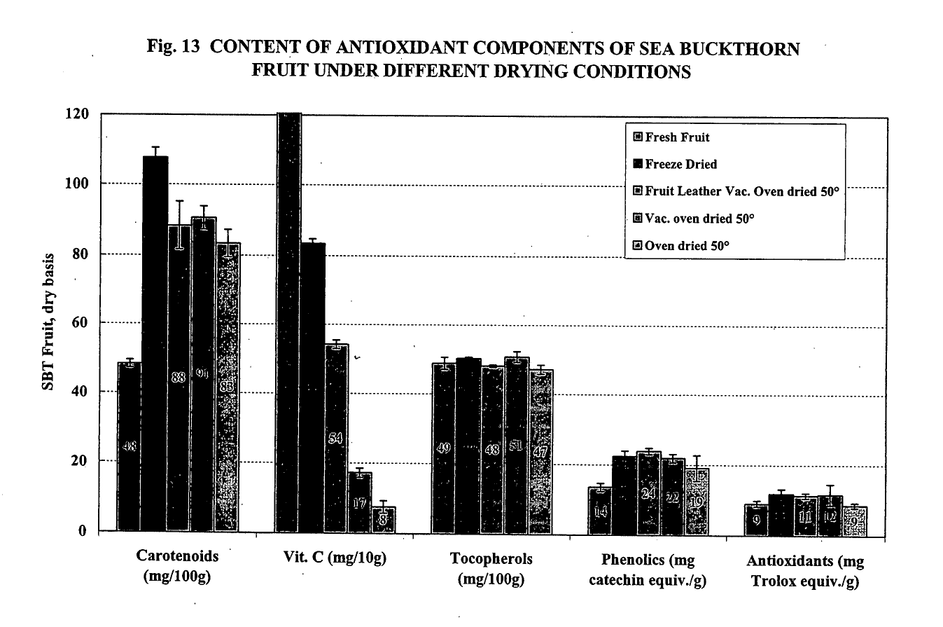

Levels of certain bioactive agents in sea buckthorn as compared to other berries are shown in FIGS. 11 and 12. Figs, 11A-B show that the level of vitamins C and E are the highest in sea buckthorn as compared to other berries. FIGS. 12A-B show that sea buckthorn berries have significant levels of quercetin and flavonols.

### Example 5

### Optimal Drying Conditions for Sea Buckthorn Leaves and Berries

Contents of total phenolics, carotenoids, vitamin E, ascorbic acid and antioxidant activity in sea buckthorn leaves vary under different drying conditions. Leaves from female trees have higher antioxidant activity that those from male trees under all drying conditions due to higher phenolic content. Oven drying at 60° C. or freeze drying is optimal for preserving antioxidant properties. Feeze-drying may further arrest enzymatic degradation of anti-oxidant compounds while the high 60° C. temperature may inactivate such enzymes. The antioxidant/phenolics ratios between male and female leaves vary under same drying conditions suggesting a qualitative difference between phenolics in male and female leaves.

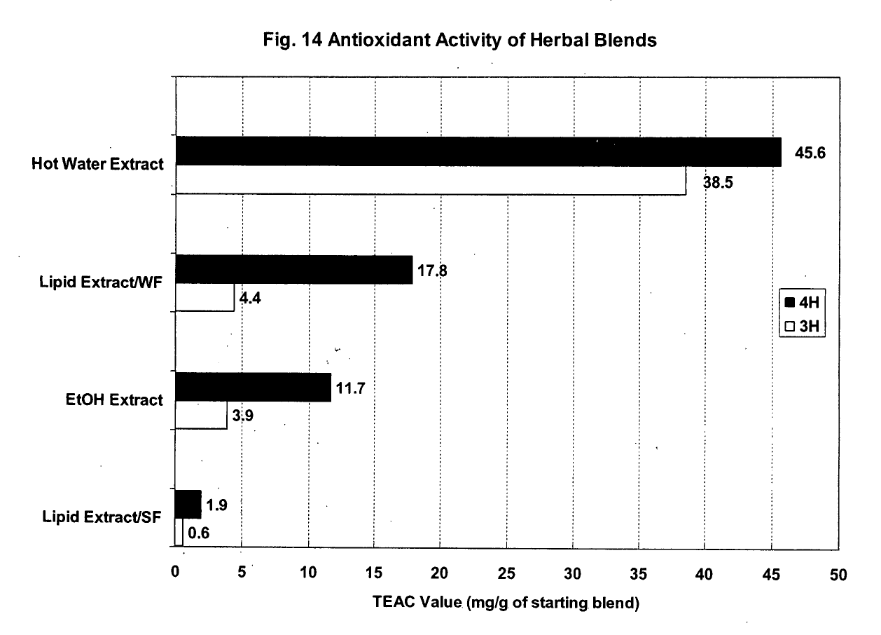

FIG. 13 shows the levels of various antioxidant compounds of sea buckthorn fruit that are recivered by different drying methods. With the exception of vitamin C, levels of all antioxidants are increased by drying. Freeze drying conditions appear optimal for sustenance of antioxidant activities.

### Example 5

### Anti-Oxidant Activity of Sea Buckthorn in Combination with Botanical Extracts

Blends of botanical extracts comprising two or more of sea buckthorn berry, sea buckthorn leaf, Panax quinquefolium (Pq), Ganoderma lucidum, Salvia miltiorrhiza and Scutellaria barbata are tested for anti-oxidant property. Blends of hot water extracts comprising two or more of Hippophae rhamnoides (Hr) berry, Hr leaf, Pq, Ganoderma lucidum, Salvia miltiorrhiza and Scutellaria barbata were tested for anti-oxidant properties.

The standard of comparison is Trolox (a water-soluble analog of vitamin E), and the relative anti-oxidant activity of the extract is defined as Trolox Equivalents (TE). In another method, the standard of comparison is Quercetin (a flavonoid), and the relative anti-oxidant activity is defined as Quercetin Equivalents.

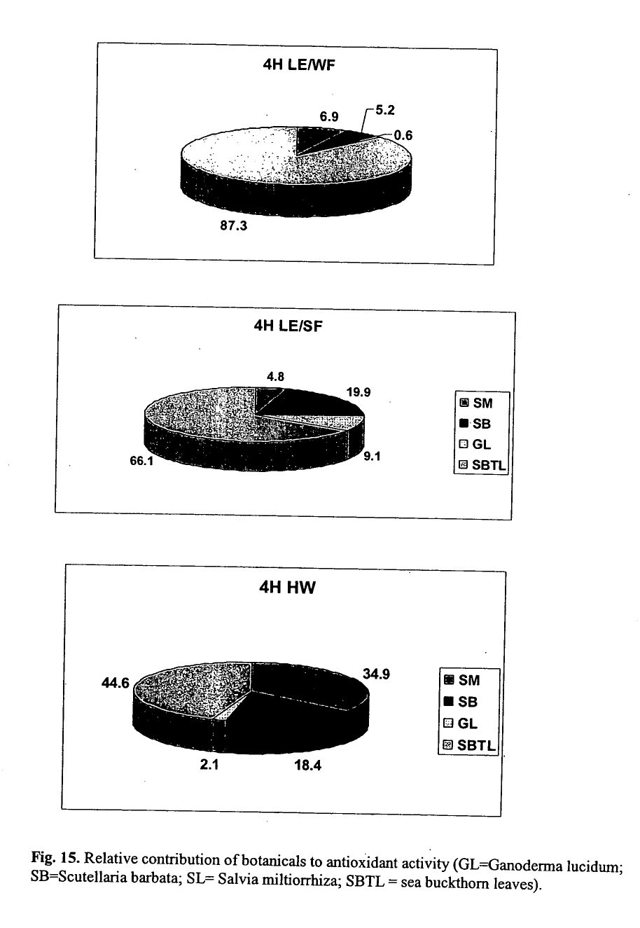

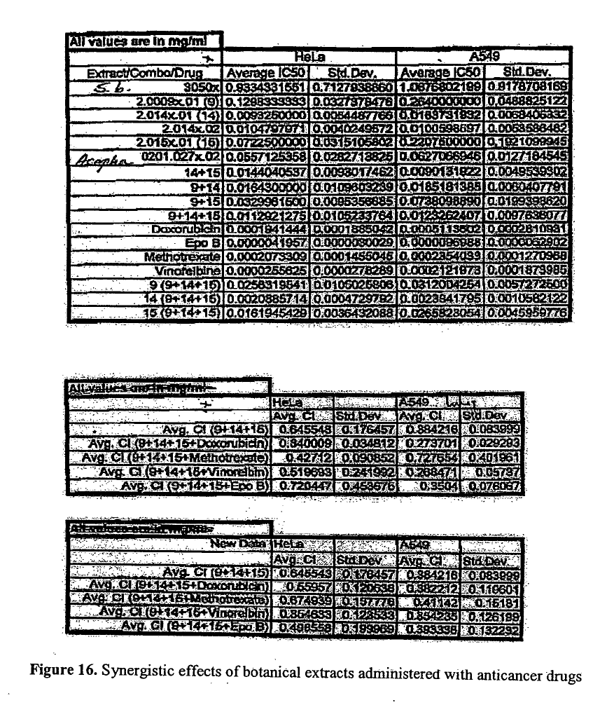

FIG. 14 shows the antioxidant activities of botanical blends under different extraction procedures. 3H represents Ganoderma lucidum, Salvia miltiorrhiza and Scutellaria barbata and 4H further includes Hippophae rhamnoides. Significant contribution towards antioxidant levels by Hippophae rhamnoides are observed under all extraction conditions. Sea buckthorn leaf was found to be responsible for nearly 50% on the anti-oxidant activity of the entire blend under both systems of measurement as shown in FIG. 15.

### Example 6

### Synergistic Effect of Botanical Extracts Administered with Anticancer Drugs

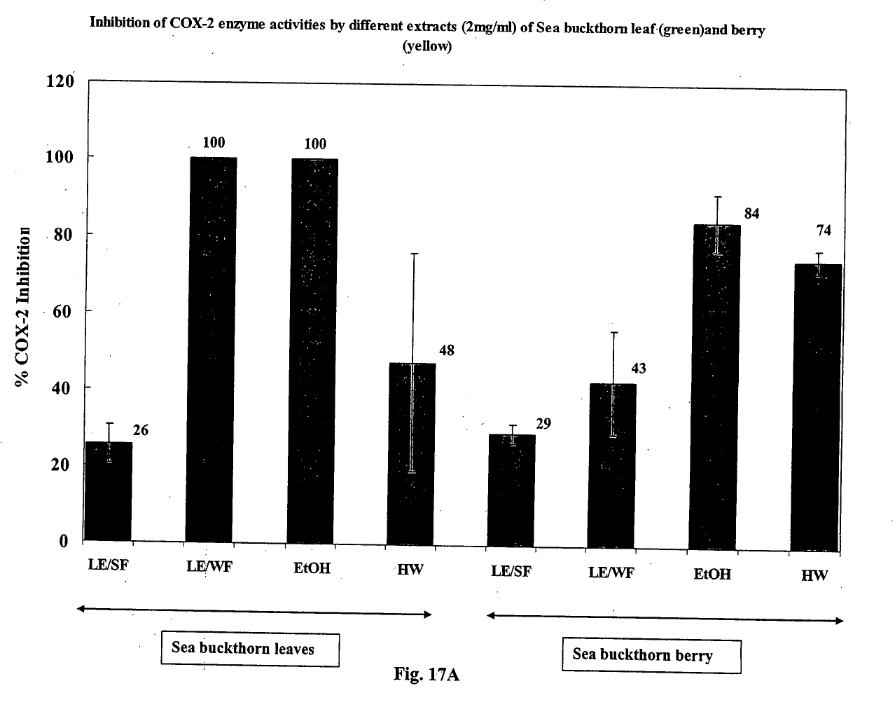

Copending U.S. application Ser. No. ______ (Attorney Docket No. 544302000100; the disclosure of which is incorporated herein in its entirety) is directed to all combinations of the three botanical extracts of Salvia miltiorrhiza (#14), Ganoderma lucidum (#9), and Scutellaria barbata (#15) which synergistically inhibit proliferation of human cancer cells such as lung cancer cells, breast cancer cells, prostate cancer cells and colon cancer cells. A 1:1:1 mixture of extracts of the individual botanicals Ganoderma lucidum, Salvia miltiorrhiza, Scutellaria barbata (“Aneustat™”; item #s 9, 14 and 15 in FIG. 16) was tested for synergistic enhancement of the efficacy of anticancer drugs in inhibiting cancer cell growth. The IC₅₀ of each botanical extract, ACAPHA, sea buckthorn (# 3050) and the anti-cancer drugs doxorubicin, Epo B, methotrexate and vinorelbine, individually and in combination, was determined in HeLa and A549 lung cancer cell lines as shown in the top panel of FIG. 16.

Synergism was measured as combination index (CI) values where values of 0.7 or less is considered to be significant levels of synergism. The middle panel of FIG. 16 shows an average of results with a fixed concentration of the three botanical extracts and varying concentrations of doxorubicin, Epo B, methotrexate and vinorelbine. Combinations of the three botanical extracts with chemotherapeutic agnets are known as Aneutox™. The bottom panel of FIG. 16 shows averages of results with a fixed ration of concentrations of the three botanical extracts and those of doxorubicin, Epo B, methotrexate and vinorelbine. The mixtures were serially diluted 2×, 4×, 8×, etc. to determine the average values.

In one embodiment, compositions of the present invention comprise effective amounts of extracts of Ganoderma lucidum, Scutellaria barbata, Salvia miltiorrhiza, and Hippophae rhamnoides (sea buckthorn) that exhibit cytostatic effects for use in inhibiting further growth of pre-existing cancer cells by exhibiting one or more properties of (i) boosting the immune system, (ii) reducing oxidative damage to cells and tissues, (iii) reducing inflammation, (iv) arresing proliferation of cells in certain stages of the cell cycle, (v) anti-oxidant activity, and (vi) anti-mutagenic effects against further exposure to carcinogens and mutagens.

### Example 7

### Cox-2/Cox-1 Inhibition by Sea Buckthorn Extracts

Cyclooxygenase (Cox) is an enzyme naturally present in our body. Cox-2 is an enzyme that is necessary for inducing pain. Nonsteroidal anti-inflammatory drugs (NSAIDs) are widely used in treating pain and the signs and symptoms of arthritis because of their analgesic and anti-inflammatory activity. It is accepted that common NSAIDs work by blocking the activity of cyclooxygenase (COX), also known as prostaglandin G/H synthase (PGHS), the enzyme that converts arachidonic acid into prostanoids. Recently, two forms of COX were identified, a constitutive isoform (COX-1) and an inducible isoform (COX-2) of which expression is upregulated at sites of inflammation (Vane, J. R.; Mitchell, J. A.; Appleton, I.; Tomlinson, A.; Bishop-Bailey, D.; Croxtoll, J.; Willoughby, D. A. Proc. Natnl. Acad. Sci. USA, 1994, 91, 2046). COX-1 is thought to play a physiological role and to be responsible for gastrointestinal and renal protection. On the other hand, COX-2 appears to play a pathological role and to be the predominant isoform present in inflammation conditions. The Cox2 enzyme is specific for inflammation, and Cox2 inhibitors (such as Celebrex®, Vioxx®) were recently approved by the FDA.

A large body of evidence suggests that cyclooxygenase-2 (COX-2) is important in gastrointestinal cancer. Levels of COX-2 mRNA were increased by >60-fold in pancreatic cancer compared to adjacent nontumorous tissue. (Tucker et al., Cancer Res. Mar. 1, 1999;59(5):987-990.) Cyclooxygenase-2 (COX-2) was overexpressed in squamous cell carcinoma of the head and neck (HNSCC) but was undetectable in normal oral mucosa from healthy subjects. (Chan et al., Cancer Res. Mar. 1, 1999;59(5):991-994). There is now increasing evidence that a constitutive expression of COX-2 plays a role in development and progression of malignant epithelial tumors. (Denkert et al Cancer Res. Jan. 1, 2001;61(1):303-308.) Taken together, these results suggest that COX-2 may be a target for the prevention or treatment of cancer.

The anti-inflammatory assays for COX-2 inhibitory activity were conducted using prostaglandin endoperoxide H synthase-1 and -2 isozymes (PGHS-1, and -2) based on their ability to convert arachidonic acid to prostaglandins (PGs). The positive controls used in this experiment are aspirin and celebrex. A preferred COX-2 inhibitor would exhibit greater inhibition of COX-2 over COX-1 which is responsible for gastrointestinal and renal protection.

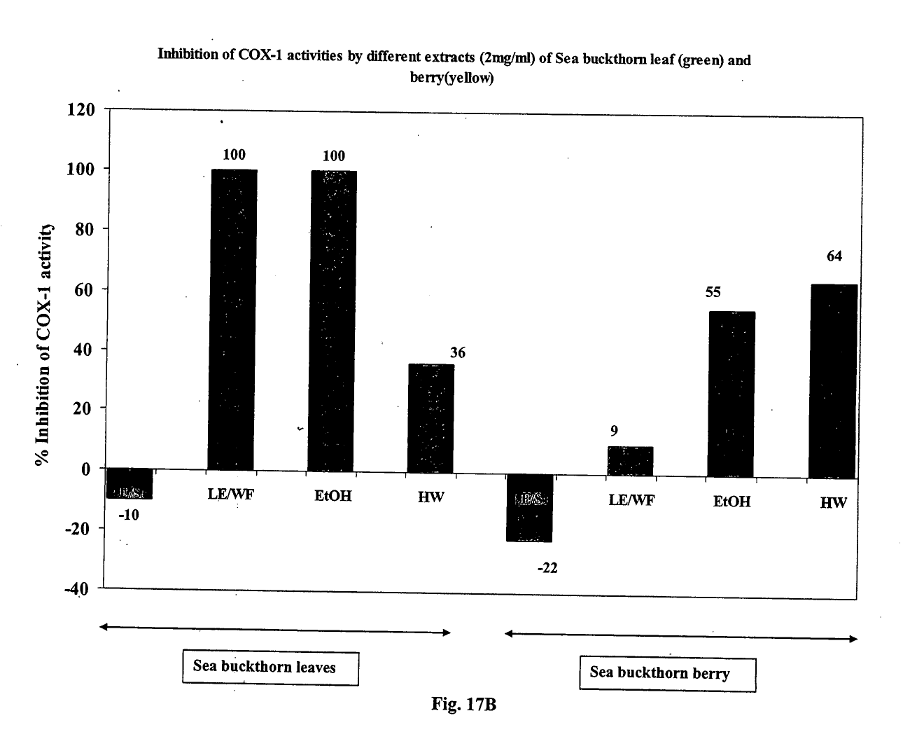

Inhibition was measured by COX-1 and COX-2 ELISA assay kits (Cayman Chemical Co., Ann Arbor, Mich.). Commercial anti-inflammatory drug aspirin inhibits COX-1 by 58% and COX-2 by 42%, while celebrex inhibits COX-1 by 46% and COX-2 by 85%. FIG. 17A shows the potencies for inhibition of COX-2 by different extracts of sea buckthorn leaves and berries at 2 mg/ml concentration. FIG. 17B shows inhibition of COX-1 by different extracts of sea buckthorn leaves and berries at 2 mg/ml concentration. Sea buckthorn leaf and berry extracts were measured separately. 80% ethanol (EtOH) and hot water (HW) extracts of sea buckthorn berry exhibit strong COX-2 and COX-1 inhibitory activities comparable to celebrex (COX-2) and aspirin (COX-1). The lipid extract/water fraction (LE/WF) and lipid extract/solvent fraction (LE/SF) of the sea buckthorn berry show very weak inhibition of COX-1 while still displaying significant inhibition of COX-2 activity. LE/WF and EtOH extracts of sea buckthorn leaves show potent inhibition of both COX-1 and COX-2 while HW and LE/WF extracts of sea buckthorn leaves show lesser but preferential inhibition of COX-2 over COX-1. LE/SF extracts of both leaf and berry show low to moderate (less than 30%) inhibition of COX-2 while displaying some activation of COX-1 activity.

All publications and patent applications cited in this specification are herein incorporated in their entirety as if each individual publication or patent application are specifically and individually indicated to be incorporated by reference.

Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

1. A method for alleviating a human cancer condition comprising:
administering to an individual at risk of developing a cancer, a prophylactically effective amount of a composition comprising an extract of Hippophae rhamnoides.

2. The method according to claim 1, wherein the composition comprises an extract of Hippophae rhamnoides leaves.

3. The method according to claim 1, wherein the composition comprises an extract of Hippophae rhamnoides berries.

4. The method according to claim 1, wherein the Hippophae rhamnoides is dried prior to extraction.

5. The method according to claim 4, wherein the Hippophae rhamnoides is dried by (a) freeze drying, or (b) oven drying at 60° C.

6. A method for inhibiting COX-2 enzyme activity comprising:
administering an effective amount of a composition comprising an extract of Hippophae rhamnoides for inhibiting COX-2.

7. The method according to claim 6, wherein inhibition of COX-2 activity by the composition is significantly higher than inhibition of COX-1 activity.

8. The method according to claim 7, further wherein inhibition of COX-2 activity by the composition is 1.5 times higher than inhibition of COX-1 activity.

9. The method according to claim 6, wherein COX-2 activity is inhibited and COX-1 activity is increased.

10. The method according to claim 6, wherein the composition comprises one or more of extracts of Hippophae rhamnoides berries, Hippophae rhamnoides berry, or Hippophae rhamnoides seeds.

11. The method according to claim 10 wherein Hippophae rhamnoides is dried prior to extraction.

12. The method according to claim 10 wherein the extract is an aqueous fraction of an organic extract.

13. The method according to claim 10 wherein the extract is a solvent fraction of an organic extract.

14. The method according to claim 10 wherein the extract is an ethanol or hot water extract of Hippophae rhamnoides berry.

15. The method according to claim 1, further comprising an extract of Camellia sinensis (green tea).

16. The method according to claim 1, further comprising one or more of an extract of Ganoderma lucidum, an extract of Salvia miltiorrhiza and an extract of Scutellaria barbata.

17. A method for alleviating a human cancer condition comprising, administering to an individual at an early stage of cancer:
(a) a therapeutically effective amount of a composition comprising an extract of Hippophae rhamnoides; and
(b) one or more extracts of Ganoderma lucidum, Scutellaria barbata, and Salvia miltiorrhiza.

18. The method according to claim 17, wherein the composition further comprises (c) a therapeutically effective amount of at least one chemotherapeutic agent.

19. The method according to claim 17, wherein the composition comprises an extract of Hippophae rhamnoides leaves.

20. The method according to claim 17, wherein the composition comprises an extract of Hippophae rhamnoides berries.

21. The method according to claim 17, wherein the Hippophae rhamnoides is dried prior to extraction.

22. The method according to claim 21, wherein the Hippophae rhamnoides is dried by (a) freeze drying, or (b) oven drying at 60° C.

23. The method according to claim 17, wherein the extract is a hot water extract.

24. The method according to claim 17, wherein the extract is an alcohol extract.

25. The method according to claim 17, wherein the extract is an organic extract.

26. The method according to claim 25 wherein the extract is a lipid fraction of the organic extract.

27. The method according to claim 25 wherein the extract is an aqueous fraction of the organic extract.

28. The method according to claim 17, further comprising administering to the individual a therapeutically effective amount of one or more anticancer treatments selected from the group consisting of radiation therapy, chemotherapy, surgery, immunotherapy, photodynamic therapy, and a combination thereof.

29. The method according to claim 17, wherein the cancer is selected from the group consisting of lung, breast, cervical and prostate cancers.

30. The method according to claim 18, wherein the chemotherapeutic agent perturbs microtubule polymerization.

31. The method according to claim 30, wherein the chemotherapeutic agent is selected from the group consisting of paclitaxel, docetaxel, etoposide, vincristine, vinblastine, and vinorelbine.

32. The method according to claim 18, wherein the chemotherapeutic agent is selected from the group consisting of cyclophosphamide, 4-hydroperoxycyclophosphamide, thiotepa, taxol, doxorubicin, daunorubicin and neocarzinostain.

33. An anticancer composition comprising one or more extracts of Ganoderma lucidum, Scutellaria barbata, and Salvia miltiorrhiza, and a therapeutically effective amount of an extract of Hippophae rhamnoides.

34. The composition according to claim 33, further comprising a therapeutically effective amount of at least one chemotherapeutic agent.

35. The composition according to claim 34, wherein the chemotherapeutic agent perturbs microtubule polymerization.

36. The composition according to claim 34, wherein the chemotherapeutic agent is selected from the group consisting of paclitaxel, docetaxel, etoposide, vincristine, vinblastine, and vinorelbine.

37. The composition according to claim 34, wherein the chemotherapeutic agent is selected from the group consisting of cyclophosphamide, 4-hydroperoxycyclophosphamide, thiotepa, taxol, doxorubicin, daunorubicin and neocarzinostain.

38. The composition according to claim 33, wherein the composition comprises one or more extracts of Hippophae rhamnoides leaves, berries and seed.

39. The composition according to claim 33, wherein the Hippophae rhamnoides is dried prior to extraction.

40. A composition for inhibiting COX-2 enzyme activity comprising an amount of an extract of Hippophae rhamnoides effective for inhibiting COX-2.

41. The composition according to claim 40, further wherein inhibition of COX-2 activity by the composition is significantly higher than inhibition of COX-1 activity.

42. The composition according to claim 41, wherein inhibition of COX-2 activity by the composition is 1.5 times higher than inhibition of COX-1 activity.

43. The composition according to claim 41, wherein COX-2 activity is inhibited and COX-1 activity is increased.

44. The composition according to claim 40 wherein the extract is an aqueous fraction of an organic extract.

45. The composition according to claim 40 wherein the extract is a solvent fraction of an organic extract.

46. The composition according to claim 40 wherein the extract is an ethanol or hot water extract of Hippophae rhamnoides berry.
